(19) United States
(12) Patent Application Publication
(10) Pub. No.: US 2008/0138806 A1
(43) Pub. Date: Jun. 12, 2008

(54) Biomarkers and detection methods for gastric diseases

(75) Inventors: Lu-Ping Chow, Taipei City (TW); Yu-Fen Lin, Tainan County (TW); Jaw-Town Lin, Taipei City (TW); Ming-Shiang Wu, Taipei City (TW)

Correspondence Address: BACON & THOMAS, PLLC, 625 SLATERS LANE, FOURTH FLOOR, ALEXANDRIA, VA, 22314, US

(73) Assignee: National Taiwan University, Taipei City (TW)

(21) Appl. No.: 11/698,820

(22) Filed: Jan. 29, 2007

(30) Foreign Application Priority Data
Dec. 8, 2006 (TW) 95145918

(51) Int. Cl. C12Q 1/68; C07H 21/04; G01N 33/53; C07K 14/00

(52) U.S. Cl. 435/6; 435/7.21; 435/7.92; 530/350; 530/388.1; 536/23.1

(57) ABSTRACT

The present invention provides a biomarker for detecting gastric diseases, especially gastric cancer selected from: a nucleic acid sequence of GroES, complementary strand, or derivatives thereof or an amino acid sequence of GroES, derivatives, fragments or variants thereof or antibodies against said amino acid sequences or combinations thereof, yet provides a kit for detecting gastric cancer by use of above-mentioned biomarkers and a detection method.

### BACKGROUND OF THE INVENTION

1. Field of the Invention

The present invention is related to detecting Helicobacter pylori-related gastric diseases by GroES protein or nucleic acid of Helicobacter pylori.

2. Description of the Prior Art

Helicobacter pylori causes chronic active gastritis, gastric ulcer, duodenal ulcer (DU) (1,2) and is strongly associated with the development of gastric cancer (GC) (3,4). Despite its decreasing incidence and mortality rate, GC is still the second most common cause of cancer-related deaths worldwide (5). In addition to host and environmental factors, chronic infection with H. pylori is regarded as a major cause of GC. Case-control studies have suggested a correlation between H. pylori seropositivity and GC. H. pylori seropositive patients have a 2.1- to 16.7-fold higher risk of developing GC than seronegative patients (3,4), and H. pylori infection is found in the majority (more than 70%) of GC patients (6,7).

Clinically, DU and GC are considered to be divergent entities. While acid production increases the risk of DU, it is reduced in patients with GC (8). Furthermore, DU is associated with a lower risk of developing GC (6,9); this finding may be attributed to the fact that DU patients have antral-predominant gastritis, in contrast to the corpus-predominant atrophic gastritis characterized as a precursor of GC (10). Recently, two studies reported the identification of candidate antigens of H. pylori associated with DU and GC by comparing the profiles of 2D-immunoblots probed with DU and GC sera (11,12). In both studies, differentially recognized antigens were determined by spot intensity, which might be biased by variations in the immune response in different diseases and in different individuals. Importantly, the serological responses towards these proteins imply that these antigens are recognized, processed, or presented by human antigen-presenting cells for initiating immune response.

In addition to eliciting humoral immune responses, H. pylori infection strongly upregulates cytokine production by monocytes/macrophages (13). These immune responses are principally associated with mucosal production of IL-8, IL-6, IL-1β, and TNF-α (14,15) and with IL-8 secretion by epithelial cells (16). Serum IL-6 and IL-1β levels have been linked to the status of H. pylori-induced GC (17). IL-8 expression is associated with angiogenic events and is strongly correlated with vessel density in GC (18). Furthermore, TNF-α and IL-10 gene polymorphisms are associated with an increased risk of non-cardia GC (19). These cytokines are therefore proposed to be critical in the pathogenesis of H. pylori-associated GC (20,21).

The host response to H. pylori infection induces multiple changes within the gastric mucosa leading to the formation of GC. The balance is altered toward decreasing in apoptosis and increasing in proliferation as H. pylori infection leads to adenocarcinoma. H. pylori infection alters expression of the cell cycle regulatory protein p27^{Kip1} which confer an apoptosis-resistant phenotype (22). Expression of proto-oncogenes c-jun and c-fos is induced by H. pylori infection (23). In addition, H. pylori also activates the expression of cyclin D1 gene in gastric epithelial cells (24). Importantly, it should be noted that cytokine responses and molecular alterations to H. pylori infection depend on both host genetic background and microbial virulence. Identification of GC-associated virulence factors of H. pylori that potentially characterize pathogen-host interactions is therefore crucial for further elucidation of the pathogenesis of H. pylori-related gastroduodenal diseases.

Although prior art discovered the relation between H. pylori and GC, actually the virulence factor of H. pylori which causes GC haven't been identified yet. Thus it is helpful to find the virulence factors of H. pylori, which causes GC, as biomarkers with high accuracy. For the aim of effectively screening patients with GC, it is important to use those biomarkers to develop detection kits for GC. By using these kits, we hope that patients with GC will be detected and properly treated at an early stage.

### SUMMARY OF THE INVENTION

In need of finding biomarkers to detect gastric disease clinically, the present invention provides a biomarker for detecting gastric diseases selected from: a nucleic acid sequence of GroES, complementary strand, or derivatives thereof or an amino acid sequence of GroES, derivatives, fragments or variants thereof or antibodies against said amino acid sequences or combinations thereof.

Another object of the present invention is to provide a biomarker for detecting gastric diseases selected from: a nucleic acid sequence of SEQ ID NO:1, complementary strand, derivatives thereof or an amino acid sequence of SEQ ID NO: 2, derivatives, fragments, variants thereof or antibodies against said amino acid sequences or combinations thereof.

Yet another object of the present invention is to provide a kit for detecting gastric disease, comprising a biomarker selected from: a nucleic acid sequence of GroES, complementary strand or derivatives thereof or an amino acid sequence of GroES, derivatives, fragments or variants thereof or antibodies against said amino acid sequences or combinations thereof.

Yet another object of the present invention is to provide a method for detecting gastric cancer, comprising following steps:
(a) providing samples;
(b) providing biomarkers, selected from: a nucleic acid sequence showing SEQ ID NO:1, complementary strand, or derivatives thereof or an amino acid sequence showing SEQ ID NO:2, derivatives, fragments or variants thereof or antibodies against said amino acid sequences or combinations thereof;
(c) contacting aforesaid biomarkers with an analyte in aforesaid samples, and the analyte selected from: a nucleic acid sequence of GroES, complementary strand or derivatives thereof or an amino acid sequence of GroES, derivatives, fragments or antibodies against aforesaid amino acid sequences of GroES or combinations thereof;
(d) detecting a product which result from the biomarker contacting with the analyte in step (c).

Yet another object of the present invention is to provide a biomarker for detecting gastric cancer selected from: an amino acid sequence of GroES, derivatives, fragments, variants thereof or the antibodies against aforesaid amino acid sequences or combinations thereof.

Yet another object of the present invention is to provide a kit for detecting gastric cancer, comprising a biomarker selected from: an amino acid sequence of GroES, derivatives, fragments, variants thereof or the antibodies against aforesaid amino acid sequences or combinations thereof, and GroES is a specific protein of H. pylori. The inventors of the present invention found a gastric disease-related protein, H. pylori GroES, which is a suitable biomarker for applying to detection of gastric disease or gastric cancer in clinical.

### BRIEF DESCRIPTION OF THE DRAWINGS

FIG. 1. 2D-profiles of GC-related immunogenic proteins. An acid-glycine extract of cell surface proteins from H. pylori was separated by 2D-electrophoresis using a linear pH 3-10 gradient in the first dimension and 12.5% SDS/PAGE in the second dimension. The separated proteins were detected by silver staining (A) or were transferred to a PVDF membrane and probed with serum from a patient with GC (B) or DU (C).

FIG. 2. Human IgG binding analysis of H. pylori GroES in gastric cancer sera samples. An acid-glycine extract of cell surface proteins from H. pylori was separated by 2D-electrophoresis. The portion of the silver-stained gel and immunoblots containing GroES isoforms are shown. The 2D-immunoblots were analyzed by probing with 15 gastric cancer sera samples, respectively. The positions of GroES isoforms are indicated (arrowheads). The “mGroES” denotes the monomeric form of GroES and “dGroES” denotes the dimeric form of GroES.

FIG. 3. Characterization of native and recombinant GroES. (A) Purification of rGroES and reactivity with anti-rGroES antibodies. Proteins in the IPTG-induced M15 cell lysate (lane 1) or the purified rGroES (lanes 2 and 3) were separated by 12.5% SDS/PAGE, then stained with Coomassie Blue (lanes 1 and 2) or immunoblotted with the anti-GroES polyclonal antibodies (lane 3). (B) 2D-immunoblots of acid-glycine extract from H. pylori probed with serum from a GC patient (left) or with anti-GroES antibodies (right). The lower box marks the monomeric form of GroES with a molecular weight ranging from 14 to 21 kDa, while the upper box indicates the dimeric form. (C) Western blot analysis using anti-GroES antibodies showing the presence of secreted GroES in the culture medium of H. pylori collected after 48-72 h incubation (lane 2), but not in medium only (lane 1) (* and ** denote the monomeric and dimeric forms of GroES, respectively).

FIG. 4. GroES stimulates inflammatory responses in PBMC. (A) PBMC were treated with rGroES (5 mg/ml) for 4 h, then RT-PCR was used to detect mRNAs for IL-8, IL-6, GM-CSF, IL-1b, TNF-α, COX-2, and GAPDH (loading control). PBMC were incubated with various concentrations of rGroES for 24 h, then protein levels of IL-8 (B,G), IL-6 (C), GM-CSF (D), IL-1β(E), or TNF-α (F) in the culture supernatant were quantified by ELISA. (G) rGroES and LPS were first digested with proteinase K (PK) and the PK inactivated, then the mixtures were incubated with PBMC as described above (rGroES and LPS 5 mg/ml and 1 mg/ml, respectively) and IL-8 measured in the culture supernatant. (H) Western blot analysis of COX-2 protein expression. PBMC were incubated with rGroES for 24 h, and then the cell lysate was examined for COX-2 and β-actin (loading control) by Western blotting. (I) PGE2 secretion into the culture medium of PBMC treated for 24 h with rGroES. All ELISA experiments were carried out in triplicates; the results are shown as mean±SD. Student's t test was used for the statistical evaluation (*P<0.05, **P<0.01 vs. control).

FIG. 5. GroES causes potential neoplastic changes in KATO-III cells. rGroES induces expression of pro-inflammatory cytokine genes and production of IL-8 protein. (A) Cells were treated with rGroES (5 mg/ml) for 6 h, and then RT-PCR was used to examine levels of mRNAs for IL-8, IL-6, GM-CSF, IL-1β, TNF-α, and GAPDH. (B) Cells were treated with rGroES for 24 h, and then IL-8 protein in the culture supernatant was measured by ELISA. (C) rGroES stimulates cell growth. Cells were treated with rGroES for 24 h, and then the number of viable cells was measured by a MTS assay. ELISA and cell proliferation experiments were carried out in triplicates; the results are shown as the mean±SD. Student's t test was used for statistical evaluation (*P<0.05, **P<0.01 vs. control). (D) Expression of the proto-oncogenes, c-jun and c-fos, is induced by rGroES. Cells were treated with rGroES (5 mg/ml) for 6 h, and then RT-PCR was used to detect mRNAs for c-jun, c-fos, and GAPDH. (E) GroES induces expression of cell cycle-related molecules favoring cell proliferation. Cells were treated with rGroES (5 mg/ml) for 12 h and the protein levels of cyclin D1, p27^{Kip1}, and β-actin were examined by Western blotting.

FIG. 6. Comparing the effects on PBMC and KATO-III cells between GroES and FlaG. PBMC (A) and KATO-III cells (B) were treated with 5 mg/ml of each recombinant protein for 24 h, respectively. ELISA measured protein levels of IL-8, IL-6, GM-CSF, IL-1β, TNF-α and PGE2 in the culture supernatant. (C) KATO-III cells were treated with 5 mg/ml of each recombinant protein for 6-48 h, and then the number of viable cells was measured by a MTS assay. ELISA and cell proliferation experiments were carried out in triplicates; the results are shown as the mean±SD. Student's t test was used for statistical evaluation (*P<0.05, **P<0.01 vs. control).

### DETAILED DESCRIPTION OF THE INVENTION

In the present invention, we used a proteomics approach to identify GC-related antigens of H. pylori by comparing profiles of 2D-immunoblots probed with DU and GC sera. Here, we report the identification of a novel GC-related antigen, GroES. GroES enhances the production by PBMC of pro-inflammatory cytokines associated with H. pylori-induced GC. Moreover, treatment of KATO-III, a gastric carcinoma cell line, with GroES leads to cell growth and upregulation of marker proteins associated with cell proliferation. Taken together, these results suggest the promoting role of GroES in GC development. Furthermore, our report presents a method for identifying of novel GC-related H. pylori antigens that should help elucidate how these antigens contribute to the inflammation and neoplastic changes induced by this bacterium.

One of the object is to provides a biomarker for detecting gastric diseases such as gastric cancer selected from: a nucleic acid sequence of GroES, complementary strand or derivatives thereof or an amino acid sequence of GroES, derivatives, fragments, variants thereof or the antibodies against said amino acid sequences or combinations thereof. GroES is a specific protein in H. pylori.

Preferably, sequence in said nucleic acid sequence of GroES is SEQ ID NO:1 and the sequence in aforesaid amino acid sequences of GroES is SEQ ID NO:2.

Aforesaid variants have more than 80% similarity with the amino acid sequence of SEQ ID NO:2

Aforesaid derivatives means the nucleic acid or the complement strand which 3′ or 5′ terminal is modified with other nucleic acid showing sequence homology with SEQ ID NO:1 greater than 90%.

Another object of the present invention is to provide a kit for detecting gastric disease, comprising a biomarker selected from: a nucleic acid sequence of GroES, complementary strand or derivatives thereof or an amino acid sequence of GroES, derivatives, fragments or variants thereof or antibodies against said amino acid sequences or combinations thereof. GroES is a specific protein of H. pylori.

Preferably, sequence in aforesaid nucleic acid sequence of GroES is SEQ ID NO:1 and the sequence in aforesaid amino acid sequences of GroES is SEQ ID NO:2.

Preferably, the kit can further comprises a second antibody which can recognize any amino acid sequences showing SEQ ID NO:2, derivatives, fragments, variants thereof or secondary antibodies against said amino acid sequences or combinations thereof.

Yet another object of the present invention is to provide a method for detecting gastric cancer, comprising following steps:
(a) providing samples;
(b) providing biomarkers, selected from: a nucleic acid sequence showing SEQ ID NO:1, complementary strand, or derivatives thereof or an amino acid sequence showing SEQ ID NO:2, derivatives, fragments or variants thereof or antibodies against said amino acid sequences or combinations thereof;
(c) contacting aforesaid biomarkers with analytes in aforesaid samples, and the analyte selected from: a nucleic acid sequence of GroES, complementary strand or derivatives thereof or an amino acid sequence of GroES, derivatives, fragments or variants thereof or antibodies against said amino acid sequences or combinations thereof;
(d) detecting products which result from the biomarkers contacting with the analytes in step (c).

The sample can be, but not limited from serum, saliva and stomach tissue.

Preferably, the biomarker can be further immobilized on substrate, for example, but not limited to membranes microplates and biochips.

Preferably, the sample is selectively labelled with fluorescence markers in step (a).

Preferably, the method can further comprise a step which utilizing secondary antibody to recognize corresponding antibody before step (d).

“detecting product” in step (d) can be, but not limited to ELISA (enzyme-linked immunosorbent assay), RIA (radioimmunoassay), western blot or immunofluorescence assay.

“detecting product” in step (d) can be, but not limited to RT-PCR (reverse transcriptase-polymerase chain reaction) or in situ hybridization.

Yet another object of the present invention is to provide a biomarker for detecting gastric cancer selected from: an amino acid sequence of GroES, derivatives, fragments, variants thereof or the antibodies against said amino acid sequences or combinations thereof. GroES is a specific protein of H. pylori.

Preferably, the sequence in aforesaid amino acid sequences of GroES is SEQ ID NO:2.

Aforesaid variant and any one of the amino acid sequences have more than 80% similarity with the amino acid sequence of SEQ ID. NO.:2.

Yet another object of the present invention is to provide a kit for detecting gastric cancer, comprising a biomarker selected from: an amino acid sequence of GroES, derivatives, fragments, variants thereof or the antibodies against aforesaid amino acid sequences or combinations thereof, and GroES is a specific protein of H. pylori.

Preferably, the sequence in said amino acid sequences of GroES is SEQ ID NO:2.

Preferably, the kit can further comprises a second antibody which can recognize any amino acid sequences showing SEQ ID NO:2, derivatives, fragments, variants thereof or antibodies against aforesaid amino acid sequences or combinations thereof.

The present invention uses nucleic acid sequence or amino acid sequence of GroES as biomarkers, which can effectively detect H. pylori-related gastric disease, especially gastric cancer, and supply information for treatment clinically.

The advantages of the present invention are further depicted with the illustration of examples. The following is a description of the exemplary case of carrying out the biomarker, GroES of H. pylori provided by the invention for detecting gastric diseases. This exemplary case is not to be taken in a limiting sense, but is made merely for the purpose of further illustrating the materials and methods for practicing the present invention.

### EXAMPLES

### Material and Method

Bacterial Strain and Culture Conditions—H. pylori strain HC5 was isolated from endoscopic biopsy sample from the stomach of a patient with GC at the National Taiwan University Hospital. The bacteria were cultured on a BBL™ Stacker™ plate (BD Biosciences, Palo Alto, Calif.) at 37° C. under microaerobic conditions. Liquid cultures were grown in flasks containing Brucella broth (Difco Laboratories, Detroit, Mich.) supplemented with 10% fetal bovine serum (FBS, Gibco, Grand Island, N.Y.), vancomycin (12.5 mg/l; Sigma, St. Louis, Mo.), and amphotericin B (2.5 mg/l; Sigma) with constant agitation at 150 rpm for 48-72 h. The culture medium was centrifuged for 10 min at 1000×g and the supernatant filtered through a 0.2 mm filter (Pall, Ann Arbor, Mich.) to eliminate intact bacterial cells.
Patients and serum samples—Serum samples were prospectively collected from individuals who participated in a national project for the investigation of H. pylori and gastroduodenal disorders in Taiwan between December 1999 and December 2001. Our study protocol was approved by both the Institutional Research Board and the Department of Health, Executive Yuan, Taiwan. Patients with newly diagnosed GC (n=95) who underwent curative gastrectomy at our institution were enrolled. For the non-cancer groups, we screened subjects from health examination at clinics; all received an upper gastrointestinal endoscopic examination and showed no GC lesions. Ninety-four patients with gastritis and 124 with DU were enrolled. H. pylori status was determined by culture and/or histological examination of gastric biopsy specimens. Tumors were histologically classified into intestinal and diffuse types based on Lauren's classification (25). Tumor stage and location were determined by a combined evaluation of a special report form completed by the patient's doctor, the case record, and the pathology report. GC stage was categorized as early (tumor extent limited to the mucosa and submucosa) or advanced (tumor invasion beyond the muscularis propria), while tumor location was subdivided into antrum, body, and cardia. In addition, 32 subjects with a normal appearance of the gastric mucosa and no evidence of H. pylori infection were selected as controls. Fasting serum samples from all participants were collected, catalogued, aliquoted, and stored at −80° C. Aliquots were only thawed once prior to analysis.
Two-dimensional electrophoresis and immunoblotting—Cell surface proteins were extracted from H. pylori using an acid-glycine extraction procedure, as described previously (26). The H. pylori acid-glycine extract was precipitated using TCA (20%) and the proteins separated by two-dimensional electrophoresis, as described previously (27). Briefly, protein extract was incubated with 2-D sample buffer (8 M urea, 2% Pharmalyte pH 3-10, 60 mM DTT, 4% CHAPS, bromophenol blue), the first dimension of the 2-D gel was run on IPG strips (Immobiline DryStrip pH 3-10, 11 cm, GE Healthcare, UK) and the second dimension was run on 12.5% SDS-polyacrylamide gels. For immunodetection, the proteins on the 2-D gel were transferred to a PVDF membrane (Millipore, Bedford, Mass.), then the membrane was blocked by incubation for 1 h at room temperature in blocking buffer (26 mM Tris-HCl, 150 mM NaCl, pH 7.5, 1% skimmed milk), and incubated with serum samples from GC patients or DU patients or pooled normal sera (1:1000 in 0.05% Tween 20/blocking buffer). Horseradish peroxidase-conjugated goat anti-human IgG (Chemicon, Temecula, Calif.) was used as secondary antibody, and bound antibody was detected using 3-amino-9-ethyl-carbazole (AEC, Sigma) as substrate.
Protein identification—The individual protein spots were excised and subjected separately to in-gel tryptic digestion. Briefly, the spots were destained using 50 mM NH₄HCO₃ in 50% ACN and dried in a SpeedVac concentrator. The protein was then digested by incubation overnight at 37° C. with sequencing grade trypsin (Promega, Madison, Wis.) in 50 mM NH₄HCO₃, pH 7.8. The resulting peptides were extracted sequentially with 1% TFA and 0.1% TFA/60% ACN. The combined extracts were lyophilized and analyzed using a QSTARTM XL Q-TOF (Applied Biosystems, Framingham, Mass., USA) coupled to an UltiMate™ Nano LC system (Dionex/LC Packings, Amsterdam, Netherlands). Peak lists of MS/MS spectra were created using Mascot Search version 1.6b4 in Analyst® QS 1.1 (Applied Biosystems). Then the peak lists were uploaded to Mascot MS/MS Ions Search program (Mascot version 2.0) on the Matrix Science public web site and protein identification was performed against NCBInr database (3479934 protein entries in it at time searched). Up to two missed cleavages was allowed. Cysteine carbamidomethylation, glutamine/asparagine deamidation, and methionine oxidation were set as possible modifications. The error windows for peptide and MS/MS fragment ion mass values were 0.3 and 0.5 Da, respectively. MH₂²⁺ and MH₃³⁺ were selected as the precursor peptide charge states in the searching. The ions score more than 54 indicated a significant match. Individual score for the MS/MS spectrum of each peptide was larger than 20. From the hit lists, the protein names and locus-tag in H. pylori 26695 strain were selected and listed in Table I and Supplemental Table II.
Cloning and purification of the recombinant proteins—H. pylori was lysed followed by RNase treatment, and the genomic DNA was further purified using phenol-chloroform and precipitated with 70% ethanol. To amplify the DNA fragment containing the H. pylori groES gene by PCR, primer pairs used were listed in Supplemental Table I. PCR was performed using 35 cycles of 94° C. for 1 min, the annealing temperature for 1 min, and 72° C. for 2 min, followed by a final extension at 72° C. for 15 min. The gene fragment was cloned into the expression vector pQE30 (Qiagen, Chatsworth, Calif.) and transformed into E. coli strain M15. H. pylori FlaG clone (pQE30/SG13009) was kindly provided by Dr. Yuh-Ju Sun. For expression of recombinant proteins, cells were grown to an A₆₀₀ value of 0.6, induced with 1 mM isopropyl β-D-thiogalactoside (IPTG), and harvested after 6 h at 25° C. (for GroES) or 3 h at 37° C. (for FlaG). The soluble recombinant proteins were purified on a Ni²⁺-chelating Sepharose column (GE Healthcare). To remove endotoxin from the recombinant proteins solutions, the resin was first washed in a centrifuge tube using binding buffer (20 mM Tris-HCl, 0.5 M NaCl, 5 mM imidazole, pH 7.9) containing 1% Triton X-114 (Sigma), then loaded into a column and washed with binding buffer containing 0.1% Triton X-114 before elution of recombinant proteins. The purified recombinant proteins were dialyzed against PBS and the endotoxin content was measured using a QCL-1000 kit (BioWhittakerr, Walkersville, Md.). The final endotoxin content was about 36 EU/mg of protein.

TABLE I
Proteins of Helicobacter pylori showing higher frequency of recognition in GC
group than in DU group identified by nano LC-MS/MS analysis

| | | | Sequence | | Seropositivity (%) | | |
|---|---|---|---|---|---|---|---|
| | | Theoretical | coverage | | GC | DU | Ratio |
| Protein | Locus_tag | pI/Mᵣ (Da) | (%) | Score | n = 15 | n = 15 | (G/D)^{a} |
| ATP synthase subunit A | HP1134 | 5.41/55,235 | 38 | 1012 | 93.3 | 86.7 | 1.08 |
| Threonine synthase | HP0098 | 6.07/54,672 | 18 | 322 | 100 | 46.7 | 2.14 |
| Urease protein (UreC) | HP0075 | 6.37/49,055 | 11 | 181 | 100 | 60 | 1.67 |
| Hemolysin secretion protein | HP0599 | 5.85/48,331 | 41 | 840 | 100 | 80 | 1.25 |
| precursor (HylB) | | | | | | | |
| ATP synthase subunit B | HP1132 | 5.30/51,418 | 46 | 875 | 93.3 | 53.3 | 1.75 |
| Glutamine synthetase (GlnA) | HP0512 | 5.75/54,479 | 37 | 773 | 100 | 80 | 1.25 |
| ATP-dependent protease | HP1374 | 6.08/50,322 | 13 | 183 | 100 | 80 | 1.25 |
| ATP-binding subunit | | | | | | | |
| Elongation factor Tu (TufA) | HP1205 | 5.17/43,620 | 69 | 1065 | 93.3 | 53.3 | 1.75 |
| Rod shape-determining protein | HP1373 | 5.40/37,374 | 16 | 186 | 73.3 | 33.3 | 2.20 |
| (MreB) | | | | | | | |
| S-adenosylmethionine | HP0197 | 6.04/42,336 | 50 | 763 | 66.7 | 26.7 | 2.50 |
| synthetase | | | | | | | |
| Peptide chain release factor 1 | HP0077 | 5.44/39,563 | 16 | 168 | 73.3 | 20 | 3.67 |
| DNA-directed RNA polymerase | HP1293 | 4.97/38,456 | 60 | 677 | 53.3 | 6.7 | 7.96 |
| alpha subunit | | | | | | | |
| Elongation factor Tu (TufA) | HP1205 | 5.17/43,620 | 40 | 459 | 66.7 | 60 | 1.11 |
| Co-chaperonin GroES | HP0011 | 6.12/12,980 | 22 | 181 | 66.7 | 6.7 | 9.96 |
| Succinate dehydrogenase | HP0191 | 5.34/27,620 | 19 | 172 | 93.3 | 73.3 | 1.27 |
| Cell division inhibitor (MinD) | HP0331 | 6.11/29,247 | 22 | 248 | 93.3 | 73.3 | 1.27 |
| Response regulator | HP1043 | 5.24/25,422 | 24 | 194 | 80 | 46.7 | 1.71 |
| Response regulator (OmpR) | HP0166 | 5.27/25,840 | 26 | 241 | 73.3 | 33.3 | 2.20 |
| Membrane fusion protein (MtrC) | HP0606 | 8.80/25,941 | 42 | 435 | 46.7 | 20 | 2.34 |
| Membrane fusion protein (MtrC) | HP0606 | 8.80/25,941 | 37 | 363 | 46.7 | 20 | 2.34 |
| Response regulator (OmpR) | HP0166 | 5.27/25,840 | 34 | 184 | 80 | 33.3 | 2.40 |
| Outer membrane protein | HP0923 | 5.84/20,011 | 37 | 326 | 53.3 | 40 | 1.33 |
| (Omp22) | | | | | | | |
| Biotin carboxyl carrier protein | HP0371 | 5.39/17,122 | 35 | 136 | 53.3 | 26.7 | 2.00 |
| (FabE) | | | | | | | |
| Co-chaperonin GroES | HP0011 | 6.12/12,980 | 28 | 203 | 66.7 | 6.7 | 9.96 |

^{a}Ratio (G/D): GC seropositivity vs. DU seropositivity

Supplemental Table I
Primer sequences used for the amplification of target genes

| | | |
|---|---|---|
| Target gene | Primer sequence [sense (+), anti-sense (−)] | T^{a} (° C.) |

| | | |
|---|---|---|
| H. pylorigroES | (+) 5′-GGATGCATGAAGTTTCAGCCATTAGGAGA-3′ | 55 |
| | (−) 5′-GGTACCTTAGTGTTTTTTGTGATCATGACA-3′ | |
| IL-1β | (+) 5′-ATA AGC CCA CTC TAC AGC T-3′ | 60 |
| | (−) 5′-ATT GGC CCT GAA AGG AGA GA-3′ | |
| IL-6 | (+) 5′-GTA CCC CCA GGA GAA GAT TC-3′ | 60 |
| | (−) 5′-CAA ACT GCA TAG CCA CTT TC-3′ | |
| IL-8 | (+) 5′-GCT TTC TGA TGG AAG AGA GC-3′ | 60 |
| | (−) 5′-GGC ACA GTG GAA CAA GGA CT-3′ | |
| IL-12 | (+) 5′-TCA CAA AGG AGG CGA GGT TC-3′ | 60 |
| | (−) 5′-TGA ACG GCA TCC ACC ATG AC-3′ | |
| GM-CSF | (+) 5′-TGG CTG CAG AGC CTG CTG CTC-3′ | 60 |
| | (−) 5′-TCA CTC CTG GAC TGG CTC CCA GCA G-3′ | |
| TNF-α | (+) 5′-GCC GGG CCA ATG CCC TCC TGG CCA A-3′ | 60 |
| | (−) 5′-GTA GAC CTG CCC AGA CTC GGC AAA-3′ | |
| IFN-γ | (+) 5′-ATA ATG CAG AGC CAA ATT GTC TC-3′ | 60 |
| | (−) 5′-CTG GGA TGC TCT TCG ACC TC-3′ | |
| COX-2 | (+) 5′-TTC AAA TGA GAT TGT GGG AAA ATT GCT-3′ | 60 |
| | (−) 5′-AGA TCA TCT CTG CCT GAG TAT CTT-3′ | |
| c-jun | (+) 5′-GGA AAC GAC CTT CTA TGA CGA GCC C-3′ | 56 |
| | (−) 5′-GAA CCC CTC CTG CTC ATC TGT CAG G-3′ | |
| c-fos | (+) 5′-ATG ATG TTC TCG GGC TTC-3′ | 48 |
| | (−) 5′-CTC TCC TGC CAA TGC TCT GC-3′ | |
| GAPDH | (+) 5′-GTC TTC ACC AAC CAT GGA GAA GGC T-3′ | 60 |
| | (−) 5′-CAT GCC AGT GAG CTT CCC GTT CA-3′ | |

^{a}Annealing temperature

Preparation of polyclonal anti-GroES antibodies—New Zealand White rabbits were injected intradermally with 500 μg of purified recombinant GroES (rGroES) in 1 ml of PBS with 1 ml of complete Freund's adjuvant (Difco). Boosters of 500 μg in 1 ml of PBS emulsified with 1 ml of Freund's incomplete adjuvant (Sigma) were given intradermally at weeks 3 and 6, then the rabbit was bled 10 days after the last boost and the serum used for immunoblotting experiments.
Serologic study—Serum samples from patients with GC, gastritis, DU, or normal controls diluted to 1:1000 were screened for reactivity with GroES by immunoblotting. Recombinant GroES was electrophoresed on a 15% SDS-polyacrylamide gel and transferred to a PVDF membrane. Immunoblotting was performed as described above.
Statistical analysis—Statistical analysis was performed using SPSS, version 11.0. Categorical data were analyzed using the chi-squared test. The odds ratio (OR) and 95% confidence interval (CI) were calculated by logistic regression. Comparisons between tests by ELISA or MTS assay were made using Student's t test. A P value of <0.05 was considered statistically significant.
Cell culture—Heparinized venous blood was drawn from healthy volunteers and mononuclear cells isolated using Ficoll-Paque Plus (GE Healthcare) density gradient centrifugation, as recommended by the manufacturer. PBMC (1.8×10⁶ cells/ml) were cultured in RPMI 1640 medium (Gibco) with 0.1% FBS at 37° C. in 5% CO₂. A human gastric carcinoma cell line, KATO-III, was obtained from the Japan Cancer Research Bank and was maintained in RPMI 1640 medium with 10% FBS, 100 μg/ml streptomycin and penicillin at 37° C. in 5% CO2. KATO-III cells (7.3×10⁴ cells/ml) were cultured in RPMI 1640 medium with rGroES to detect cytokines or incubated for 16-18 h in RPMI 1640 medium; following serum starvation, the KATO-III cells were incubated with rGroES in RPMI 1640 for Western blot analysis.
RT-PCR—Cells were collected after 4 h (PBMC) or 6 h (KATO-III) stimulation with rGroES, and mRNAs were isolated using a QuickPrep™ Micro mRNA Purification Kit (GE Healthcare) following the manufacturer's recommendations. Reverse transcription reactions were performed according to the instruction manual for the SuperScript™ First-Strand Synthesis System for RT-PCR (Life Technologies Inc., Rockville, Md.). The resulting cDNA was used as template for PCR amplification using the primer pairs and the annealing temperature conditions listed in Supplemental Table I. PCR was performed as described above. As a loading control, a parallel PCR was carried out using a primer pair for human GAPDH.
Measurement of cytokines and PGE₂—Cells were incubated for 24 h with rGroES, then the supernatants were collected and stored at −80° C. until assayed for cytokine production. Levels of cytokines and PGE₂ in the culture supernatants were measured using Quantikin® ELISA assay kit (R & D Systems, Minneapolis, Minn.) for IL-8, IL-6, IL-1β, TNF-α, and GM-CSF or a Direct Biotrak Assay ELISA kit (GE Healthcare) for PGE₂ according to the manufacturer's instructions. All experiments were performed in triplicate. Furthermore, to verify that the cytokine release from cells was due to rGroES and not the contaminating LPS, rGroES and LPS were digested with proteinase K (PK/substrate molar ratio of 1/10) for 1 h at 37° C., then the PK was inactivated by heating at 100° C. for 10 min. PK-treated rGroES and LPS were then used to treat cells as described above.
Western blot analysis—After treatment with rGroES for 12 or 24 h, cells were treated with lysis buffer (0.6% NP-40, 0.9% NaCl, 0.1% SDS, 1 mM EDTA, 10 mM Tris-HCl, pH 7.5), followed by centrifugation at 18000 g for 15 min at 4° C. to remove cell debris. Immunoblot analysis was performed as described above. The primary antibodies used were goat anti-COX-2 (1:200, Santa Cruz Biotechnology, Santa Cruz, Calif.), and mouse anti-cyclin D1 (1:500, Santa Cruz Biotechnology), mouse anti-p27^{Kip1} (1:1000, BD Biosciences Transduction Laboratories), and mouse anti-β-actin (1:100000, CashmereBiotech, Taipei Hsien, Taiwan). The secondary antibodies used were HRP-conjugated anti-mouse IgG antibody (BD Biosciences PharMingen) or anti-goat IgG antibody (Sigma). Bound antibody was detected using ECL™ reagent (GE Healthcare), followed by exposure to X-ray film (Kodak, Rochester, N.Y.). β-actin was used as the loading control.
Cell proliferation assay—KATO-III cells (8000 cells/well) were cultured in 100 ml 0.1% FBS/RPMI 1640 medium with or without rGroES in a 96-well culture plate for 6 h, 24 h, 36 h and 48 h. The number of viable cells was measured by a MTS assay (CellTiter 96® AQᵤₑₒᵤₛ One Solution Cell Proliferation Assay, Promega). Assay was performed by adding 20 ml above reagent to each well incubated at 37° C. for 1 h and then measured at the absorbance 490 nm. Results are presented as the percentage of nontreated cells after subtracting the blank values (medium only). The experiments were performed in triplicate.

### Example 1

### Identification of Gastric Cancer-Related Antigens of H. pylori

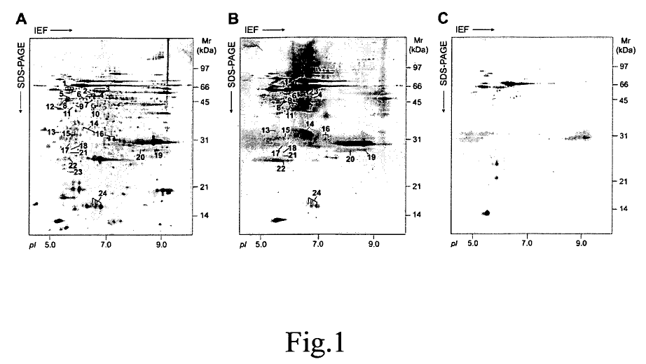

To identify candidate H. pylori antigens associated with GC, we performed 2D-SDS/PAGE on the bacterial proteins extracted with acidic glycine and compared the patterns of 2D-immunoblots probed with sera from H. pylori-infected patients with either GC or DU. Silver staining revealed a complex protein profile of the acid-glycine extract (FIG. 1A). Probing with 15 GC sera and 15 DU sera gave unique and different patterns of reactivity. Two representative immunoblots are shown in FIG. 1B (GC) and FIG. 1C (DU).

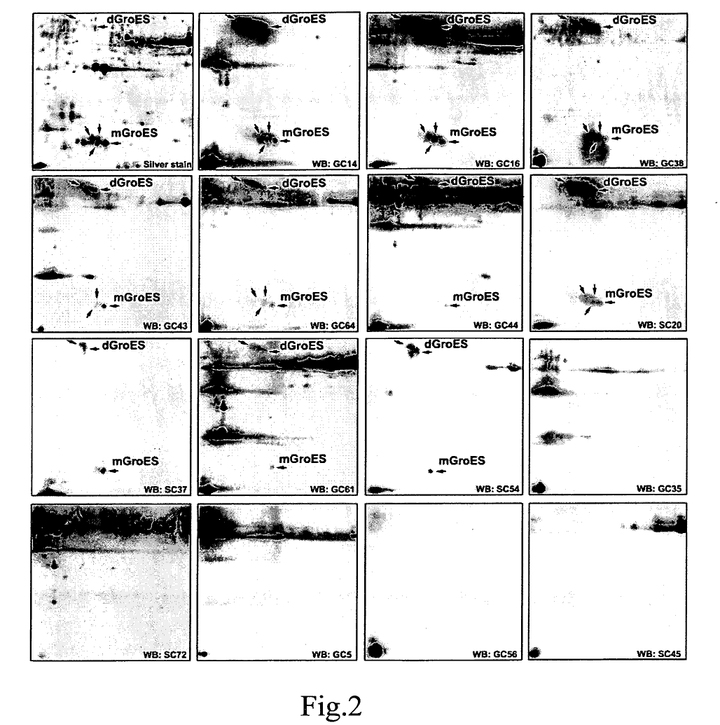

In general, the frequency of spot recognition was greater with GC sera than with DU sera. On the GC immunoblots, about 60 different reactive protein spots were detected, with molecular weights ranging from 14 to 85 kDa and pIs ranging from 4.5 to 9.5. Some of these antigenic spots were recognized by individual serum sample, but 49 spots were recognized by more than one. Comparing the antigenic protein profile of these 2D-immunoblots, 24 spots/spot groups were more frequently recognized by GC sera. The spots with differential frequencies of recognition were subsequently identified by nano-LC-MS/MS ions search and shown in Table I and Supplemental Table II. The proteins showing higher frequency of recognition in GC group (GC vs. DU seropositivity ratio>2) are threonine synthase, rod shape-determining protein, S-adenosylmethionine synthetase, peptide chain release factor 1, DNA-directed RNA polymerase alpha subunit, co-chaperonin GroES (monomeric and dimeric forms), response regulator OmpR, and membrane fusion protein. Among the identified proteins, two forms of co-chaperonin GroES monomer and dimer, indicated in FIG. 2, exhibited the highest frequency of differential recognition by GC sera (66.7%), but only one of the fifteen (6.7%) DU sera (data not shown). Therefore, co-chaperonin GroES considered as important immunogenic proteins.

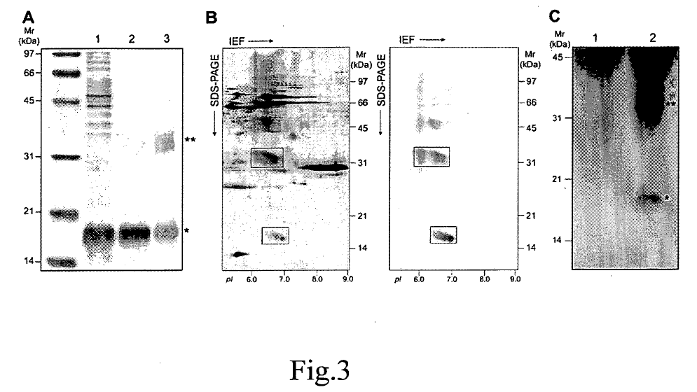

To investigate the biochemical features of GroES, we expressed recombinant His-tagged GroES fusion protein in E. coli M15 and used the purified recombinant GroES (rGroES) to generate an anti-GroES antiserum in rabbits. Recombinant GroES with an apparent molecular weight of 17 kDa was successfully expressed in E. coli M15 (FIG. 3A, lane 1). The identity of the purified rGroES (FIG. 3A, lane 2) was confirmed by nano-LC-MS/MS. Furthermore, the existence of monomeric and dimeric forms of rGroES was observed by immunoblot analysis using the anti-GroES antibodies (FIG. 3A, lane 3).

We further characterize the native GroES of H. pylori by immunoblot analysis of the 2D map of acid-glycine-extracted proteins using GC sera (FIG. 3B, left) and the anti-GroES antibodies (FIG. 3B, right). As with rGroES, we detected the presence of multimeric forms of native GroES in the H. pylori cell extract, in addition to the monomeric and dimeric forms originally identified as the GC-related antigenic spots. Using patients' sera, the dimeric form of native GroES appeared to be more prevalent than the monomeric and trimeric forms (FIG. 3B, left). Furthermore, although mainly found in the H. pylori extract, GroES was also detected in the filtered medium from H. pylori cultures, suggesting that GroES is secreted out of H. pylori (FIG. 3C).

SUPPLEMENTAL TABLE II
Identifying the immuno-reactive proteins of Helicobacter pylori showing higher frequency of recognition in
GC group by nano-LC-MS/MS analysis

| | | | | | | No. of | |
|---|---|---|---|---|---|---|---|
| | | | | | Sequence | peptides | |
| Spot | | | NCBI | | coverage | (unique/ | |
| no. | Protein | Locus_tag | accession no. | Score | (%) | matched) | Unique peptides list |
| 1 | ATP | HP1134 | gi\|18075728 | 1012 | 38 | 23/39 | K.LEEISSVIEEK.I |
| | synthase | | | | | | K.VVSYADGVAK.V |
| | subunit A | | | | | | K.VPVGDAVVGR.V |
| | | | | | | | R.VLNALGEPIDGK.G |
| | | | | | | | R.KSVHEPLQTGIK.A |
| | | | | | | | K.SVHEPLQTGIK.A |
| | | | | | | | K.AIDALVPIGR.G |
| | | | | | | | K.ESTVAQVVR.K |
| | | | | | | | R.HALIIYDDLSK.H |
| | | | | | | | R.EISLILR.R |
| | | | | | | | R.EAFPGDVFYIHSR.L |
| | | | | | | | R.LDLAQYR.E |
| | | | | | | | R.ELQAFTQFASDLDEASK.K |
| | | | | | | | R.ELQAFTQFASDLDEASKK.Q |
| | | | | | | | K.QAPYSPLPIEK.Q |
| | | | | | | | K.GFLDSVSVK.K |
| | | | | | | | K.KVVDFEEQLHPFLEAK.Y |
| | | | | | | | K.VVDFEEQLHPFLEAK.Y |
| | | | | | | | K.YPQVLEEIHTK.K |
| | | | | | | | K.KVLDKDLEAMLR.K |
| | | | | | | | K.VLDKDLEAMLR.K |
| | | | | | | | K.DLEAMLR.K |
| | | | | | | | R.KVLEEFK.L |
| 2 | Threonine | HP0098 | gi\|15644728 | 322 | 18 | 8/8 | K.KIDFIEAILNPNAPK.G |
| | synthase | | | | | | K.IDFIEAILNPNAPK.G |
| | | | | | | | K.NPAPIFALNER.L |
| | | | | | | | R.LFVQELYHGPSLAFK.D |
| | | | | | | | K.LQMVTQSASNLK.V |
| | | | | | | | K.VFGISGDFDDAQNALK.N |
| | | | | | | | K.LSVANSVNFGR.I |
| | | | | | | | K.TLVSATASYEK.F |
| 3 | Urease | HP0075 | gi\|15644705 | 181 | 11 | 5/5 | K.FFNSYGYK.L |
| | protein | | | | | | R.IVLDTANGAAYK.V |
| | (UreC) | | | | | | R.ADLGFAFDGDADR.L |
| | | | | | | | K.LLGVLGVYQK.S |
| | | | | | | | K.ELDKLEIR.H |
| 4 | Hemolysin | HP0599 | gi\|15645224 | 840 | 41 | 15/32 | K.SGNLASLNNLEEQSVHFK.E |
| | secretion | | | | | | K.ENAESVNLQGVSYSLK.S |
| | protein | | | | | | K.SQNIDGVQYFSLAK.N |
| | precursor | | | | | | K.NGEAHSTEGLGTVNK.T |
| | (HylB) | | | | | | K.TGQDIESLYEK.M |
| | | | | | | | K.MQNATSLADSLNQR.S |
| | | | | | | | R.GFAVVADEVR.K |
| | | | | | | | R.GFAVVADEVRK.L |
| | | | | | | | K.NNMIVAQAAK.Y |
| | | | | | | | K.YTIYNINNR.V |
| | | | | | | | K.LDHVVFK.N |
| | | | | | | | K.NNLYGMVFGLNSFDITSHK.N |
| | | | | | | | K.WYYEGAGK.E |
| | | | | | | | K.ENFSNTSGYR.A |
| | | | | | | | R.ALESHHASVHAEANDLVK.A |
| 5 | ATP | HP1132 | gi\|2197129 | 875 | 46 | 16/33 | K.SLVLEVAAHLGGNR.V |
| | synthase | | | | | | R.AIAMDMTEGLVR.N |
| | subunit B | | | | | | K.MIEVPVGEEVLGR.I |
| | | | | | | | K.TEMFETGIK.V |
| | | | | | | | K.VIDLLAPYSK.G |
| | | | | | | | K.VGLFGGAGVGK.T |
| | | | | | | | K.TVIIMELIHNVAYK.H |
| | | | | | | | K.HNGYSVFAGVGER.T |
| | | | | | | | R.IAFTGLTMAEYFR.D |
| | | | | | | | R.YAQSGAEMSALLGR.I |
| | | | | | | | R.IPSAVGYQPTLAGEMGK.L |
| | | | | | | | K.GIYPAVDPLDSTSR.I |
| | | | | | | | R.ILSPQMIGEK.H |
| | | | | | | | K.HYEIATGIQQVLQK.Y |
| | | | | | | | K.FLSQPFFVAEVFTGSPGK.Y |
| | | | | | | | K.YDHIPENAFYMVGSIQEVLEK.A |
| 6 | Glutamine | HP0512 | gi\|15645139 | 773 | 37 | 15/24 | K.ENEVEFVDFR.F |
| | synthetase | | | | | | K.GWQGIEHSDMILTPDLVR.Y |
| | (GlnA) | | | | | | R.SFENGVNFGHRPGK.Q |
| | | | | | | | K.VLNQVGLETFVVHHEVAQAQGEVGVK.F |
| | | | | | | | K.FGDLVEAADNVQK.L |
| | | | | | | | K.NNENLFSGETYK.G |
| | | | | | | | R.GLAAFTNASTNSYK.R |
| | | | | | | | R.GLAAFTNASTNSYKR.L |
| | | | | | | | R.LIPGYEAPSILTYSANNR.S |
| | | | | | | | K.NKIDPGEAMDINLFK.L |
| | | | | | | | K.IDPGEAMDINLFK.L |
| | | | | | | | K.LTLDEIR.E |
| | | | | | | | R.SLEEMLADK.Q |
| | | | | | | | R.SLEEMLADKQYLK.E |
| | | | | | | | K.ESQVFSEEFIQAYQSLK.F |
| 7 | ATP- | HP1374 | gi\|15645984 | 183 | 13 | 6/6 | R.IIFASNLNK.D |
| | dependent | | | | | | K.AVLDNYVIGQEQAK.K |
| | protease | | | | | | K.SNILLIGPTGSGK.T |
| | ATP- | | | | | | K.GIVFIDEIDK.I |
| | binding | | | | | | K.GIVFIDEIDKISR.L |
| | subunit | | | | | | R.TTQNVLGFTQEK.M |
| 8 | Elongation | HP1205 | gi\|15645819 | 1065 | 69 | 22/48 | R.TKPHVNIGTIGHVDHGK.T |
| | factor Tu | | | | | | K.TTLSAAISAVLSLK.G |
| | (TufA) | | | | | | K.GLAEMKDYDNIDNAPEEK.E |
| | | | | | | | K.DYDNIDNAPEEK.E |
| | | | | | | | K.DYDNIDNAPEEKER.G |
| | | | | | | | R.GITIATSHIEYETENR.H |
| | | | | | | | K.NMITGAAQMDGAILVVSAADGPMPQTR.E |
| | | | | | | | R.EHILLSR.Q |
| | | | | | | | R.QVGVPHIVVFLNK.Q |
| | | | | | | | K.QDMVDDQELLELVEMEVR.E |
| | | | | | | | R.ELLSAYEFPGDDTPIVAGSALR.A |
| | | | | | | | K.LMAEVDAYIPTPER.D |
| | | | | | | | K.LMAEVDAYIPTPERDTEK.T |
| | | | | | | | K.TFLMPVEDVFSIAGR.G |
| | | | | | | | K.TTVTGVEMFR.K |
| | | | | | | | R.KELEKGEAGDNVGVLLR.G |
| | | | | | | | K.ELEKGEAGDNVGVLLR.G |
| | | | | | | | K.GEAGDNVGVLLR.G |
| | | | | | | | K.KFEGEIYVLSK.E |
| | | | | | | | R.TTDVTGSITLPEGVEMVMPGDNVK.I |
| | | | | | | | K.ITVELISPVALELGTK.F |
| | | | | | | | R.TVGAGVVSNIIE.— |
| 9 | Rod | HP1373 | gi\|15645983 | 186 | 16 | 4/4 | K.AYDILAVGSEAK.E |
| | shape- | | | | | | R.VAGDKLDQSIVEYIR.K |
| | de- | | | | | | K.LPVYVGDEPLLAVAK.G |
| | termining | | | | | | K.GTGEAIQDLDLLSR.V |
| | protein | | | | | | |
| | (MreB) | | | | | | |
| 10 | S- | HP0197 | gi\|15644826 | 763 | 50 | 14/28 | K.DSFLFTSESVTEGHPDK.M |
| | adeno- | | | | | | K.MADQISDAVLDYIIER.D |
| | sylmethi- | | | | | | K.TSVYAPMQEIAR.E |
| | onine | | | | | | K.IGYTDALYGFDYR.S |
| | synthetase | | | | | | R.SAAVLNGVGEQSPDINQGVDR.E |
| | | | | | | | K.ETETLMPLPIHLAHQLTFALAQK.R |
| | | | | | | | R.KDNTLPFLRPDGK.S |
| | | | | | | | K.DNTLPFLRPDGK.S |
| | | | | | | | R.YENNKPVSIDTIVISTQHSPEVSQK.H |
| | | | | | | | K.EAVIEBIVYK.V |
| | | | | | | | K.FVIGGPQGDAGLTGR.K |
| | | | | | | | K.YSSAELEK.C |
| | | | | | | | K.TNKAEEIKAFFK.R |
| | | | | | | | K.AEEIKAFFK.R |
| 11 | Peptide | HP0077 | gi\|015644707 | 168 | 16 | 4/4 | K.EYLSVLENIK.E |
| | chain | | | | | | K.ELLEDKELSELAKEELK.I |
| | release | | | | | | K.DPNDDKNIYLELR.A |
| | factor 1 | | | | | | R.AGTGGDEAGIFVGDLFK.A |
| 12 | DNA- | HP1293 | gi\|4155841 | 677 | 60 | 14/36 | K.TAPLIPSEIK.V |
| | directed | | | | | | K.ISLAPFEFGYAVTLAHPIR.R |
| | RNA | | | | | | R.LLLLSSVGYAPVGLK.I |
| | poly- | | | | | | K.IEGVHHEFDSLR.G |
| | merase | | | | | | R.GVTEDVSLFIMNLK.N |
| | alpha | | | | | | K.ALVGQDSSLENQSVVVDYSFK.G |
| | subunit | | | | | | K.GMGYVPSENTR.E |
| | | | | | | | R.ELMPEGYMPLDGSFTPIK.N |
| | | | | | | | K.NVVYEIENVLVEGDPNYEK.I |
| | | | | | | | K.IIFDIETDGQIDPYK.A |
| | | | | | | | K.QLGVFGERPIANTEYSGDYAQR.D |
| | | | | | | | K.IESMNLSAR.C |
| | | | | | | | K.YVGELVLMSEEELK.G |
| | | | | | | | K.SYDEIAEK.L |
| 13 | Elongation | HP1205 | gi\|15645819 | 459 | 40 | 13/16 | R.GITIATSHIEYETENR.H |
| | factor Tu | | | | | | R.EHILLSR.Q |
| | (TufA) | | | | | | R.QVGVPHIVVFLNK.Q |
| | | | | | | | R.ELLSAYEFPGDDTPIVAGSALR.A |
| | | | | | | | K.LMAEVDAYIPTPER.D |
| | | | | | | | K.TFLMPVEDVFSIAGR.G |
| | | | | | | | K.TTVTGVEMFR.K |
| | | | | | | | K.ELEKGEAGDNVGVLLR.G |
| | | | | | | | K.GEAGDNVGVLLR.G |
| | | | | | | | K.KFEGEIYVLSK.E |
| | | | | | | | K.FEGEIYVLSK.E |
| | | | | | | | R.TTDVTGSITLPEGVEMVMPGDNVK.I |
| | | | | | | | R.TVGAGVVSNIIE.— |
| 14 | Co- | HP0011 | gi\|712830 | 181 | 36 | 5/10 | K.FQPLGER.V |
| | chaperonin | | | | | | R.LEEENKTSSGIIIPDNAK.E |
| | GroES | | | | | | K.TSSGIIIPDNAK.E |
| | | | | | | | K.EKPLMGVVK.A |
| | | | | | | | K.EGDVIAFGK.Y |
| 15 | Succinate | HP0191 | gi\|2058520 | 172 | 19 | 4/5 | K.FDPQSAVSKPHFK.E |
| | de- | | | | | | R.IEPDEAQEVFELDR.C |
| | hydrogenase | | | | | | R.FMIDSHDER.S |
| | | | | | | | K.ELPLQSSIATLR.N |
| 16 | Cell division | HP0331 | gi\|4154852 | 248 | 22 | 6/7 | M.AIVVTITSGK.G |
| | inhibitor | | | | | | R.NLDMILGLENR.I |
| | (MinD) | | | | | | R.IVYDVVDVMEK.N |
| | | | | | | | K.NLSFLAASQSK.D |
| | | | | | | | K.VAILINALR.A |
| | | | | | | | R.VIGIIDAK.S |
| 17 | Response | HP1043 | gi\|4154918 | 194 | 24 | 5/9 | K.NSVLGGEIEK.G |
| | regulator | | | | | | R.NYDLVMVSDK.N |
| | | | | | | | K.NALSFVSR.I |
| | | | | | | | K.GKPFEVLTHLAR.H |
| | | | | | | | K.MDKPLGISTVETVR.R |
| 18 | Response | HP0166 | gi\|15644795 | 241 | 26 | 6/6 | K.ALDYGADDYLPKPYDPK.E |
| | regulator | | | | | | K.KEEVSEPGDANIFR.V |
| | (OmpR) | | | | | | K.EEVSEPGDANIFR.V |
| | | | | | | | R.AEYEILSLLISK.K |
| | | | | | | | K.SIDVIIGR.L |
| | | | | | | | K.QPQYIISVR.G |
| 19 | Membrane | HP0606 | gi\|15645231 | 435 | 42 | 9/10 | K.VYAIFNVK.A |
| | fusion | | | | | | K.LTLDSTGIVDSIK.V |
| | protein | | | | | | K. KGDVLLLLYNQDK.Q |
| | (MtrC) | | | | | | K.GDVLLLLYNQDK.Q |
| | | | | | | | R.APFDGVIASK.N |
| | | | | | | | K.NIQVGEGVSANNTVLLR.L |
| | | | | | | | R.KLVIEFDSK.Y |
| | | | | | | | K.VGDTYTYSIDGDSNQHEAK.I |
| | | | | | | | K.IYPTVDENTR.K |
| 20 | Membrane | HP0606 | gi\|15645231 | 363 | 37 | 8/9 | K.VYAIFNVK.A |
| | fusion | | | | | | K.LTLDSTGIVDSIK.V |
| | protein | | | | | | K.KGDVLLLLYNQDK.Q |
| | (MtrC) | | | | | | K.GDVLLLLYNQDK.Q |
| | | | | | | | K.NIQVGEGVSANNTVLLR.L |
| | | | | | | | K.LVIEFDSK.Y |
| | | | | | | | K.VGDTYTYSIDGDSNQHEAK.I |
| | | | | | | | K.IYPTVDENTR.K |
| 21 | Response | HP0166 | gi\|15644795 | 184 | 34 | 6/6 | K.ALDYGADDYLPKPYDPK.E |
| | regulator | | | | | | K.KEEVSEPGDANIFR.V |
| | (OmpR) | | | | | | R.AEYEILSLLISK.K |
| | | | | | | | R.ESIAIESESINPESSNK.S |
| | | | | | | | K.SIDVIIGR.L |
| | | | | | | | K.QPQYIISVR.G |
| 22 | Outer | HP0923 | gi\|4098205 | 326 | 37 | 5/13 | K.HNMDKETVAGDVSAK.A |
| | membrane | | | | | | K.ESDQETLDEIVQK.A |
| | protein | | | | | | K.AKENHMQVLLEGNTDEFGSSEYNQALGVK.R |
| | (Omp22) | | | | | | K.ENHMQVLLEGNTDEFGSSEYNQALGVK.R |
| | | | | | | | K.TISFGETKPK.C |
| 23 | Biotin | HP0371 | gi\|15644999 | 136 | 35 | 4/6 | —.MNLSEIEELIK.E |
| | carboxyl | | | | | | K.LKHEHFELVLDKESAYAK.K |
| | carrier | | | | | | K.HEHFELVLDKESAYAK.K |
| | protein | | | | | | K.KEDFVLSPMVGTFYHAPSPGAEPYVK.A |
| | (FabE) | | | | | | |
| 24 | Co- | HP0011 | gi\|712830 | 203 | 28 | 4/32 | K.FQPLGER.V |
| | chaperonin | | | | | | R.LEEENKTSSGIIIPDNAK.E |
| | GroES | | | | | | K.TSSGIIIPDNAK.E |
| | | | | | | | K.EGDVIAFGK.Y |

### Example 2

### GroES Seropositivity is Related to Gastric Cancer

To examine the clinicopathological significance of GroES seropositivity in H. pylori-infected patients, a GroES immunoblot assay was performed on a series of clinical samples. A serum was defined as GroES seropositive if rGroES was recognized by serum IgG. No seropositivity was seen with any serum sample from 32 healthy persons without H. pylori infection (controls). We then examined the serum IgG response to GroES in 313H. pylori-infected patients with GC (95 patients), gastritis (94 patients), or DU (124 patients). Overall, 42.8% of the H. pylori-infected patients gave a positive response. GroES seropositivity was related to patient age, increasing from 18.8% in patients aged less than 30 years to 40.2% in patients aged 30-49 years (odds ratio (OR): 2.9, 95% confidence interval (CI): 0.8-10.9, P=0.1) and to 46.2% in patients aged more than 50 years (OR: 3.7, 95% CI: 1.0-13.4, P=0.04) (Supplemental Table III). Furthermore, the prevalence of GroES seropositivity in patients with GC, gastritis, or DU was 64.2%, 30.9%, and 35.5%, respectively. After adjustment for age difference, the GroES seropositivity in GC patients was significantly higher than that in gastritis patients (OR: 3.9, 95% CI: 2.1-7.4, P<0.001) or DU patients (OR: 2.7, 95% CI: 1.5-4.9, P<0.001). There was also a statistically significant difference in GroES seropositivity between controls and H. pylori-infected subjects, but not between patients with DU or gastritis (Table II). To further characterize the relationship between GC and GroES seropositivity, 95 GC patients were classified into several subtypes by gender, stage, histological type, and tumor location for statistical analysis of GroES positivity; the results are listed in Table III. Importantly, although gender, stage, and histological subtype had no significant effect on GroES seropositivity, GC located in the antrum exhibited a significant higher rate of GroES seropositivity than those in a non-antrum location (71.9% vs. 48.4%; OR: 2.7, 95% CI: 1.1-6.7, P=0.03).

TABLE II
Serum IgG GroES positivily in various upper gastrointestinal diseases

| | GroES | | Adjusted OR (95% CI) | | |
|---|---|---|---|---|---|
| | Positive | Negative | P value^{b} | | |
| Disease | no. (%) | no. (%) | GC | Gastritis | DU |
| GC | 61 (64.2) | 34 (35.8) | 1.0^{a} | — | — |
| (n = 95) | | | | | |
| Gastritis | 29 (30.9) | 65 (69.1) | 3.9 (2.1–7.4) | 1.0^{a} | — |
| (n = 94) | | | <0.001 | | |
| DU | 44 (35.5) | 80 (64.5) | 2.7 (1.5–4.9) | 0.8 (0.4–1.4) | — |
| (n = 124) | | | <0.001 | 0.3 | |
| Control | 0 (0) | 32 (100) | — | — | — |
| (n = 32) | | | <0.001 | <0.001 | <0.001 |

^{a}As the reference to calculate the OR.
^{b}ORs, 95% CI and P value were performed by logistic regression after controlling for age.

SUPPLEMENTAL TABLE III
Effect of age on GroES seropositivity among 313 H. pylori-infected patients

| Age group | Mean age ± SD | no. of | GroES-seropositive | | | |
|---|---|---|---|---|---|---|
| (yr) | (yr) | patients | (%) | OR^{a} | (95% CI)^{a} | P value^{a} |
| Total | 54.2 ± 14.1 | 313 | 42.8 | | | |
| 16–29 | 23.6 ± 3.5 | 16 | 18.8 | 1.0^{b} | | |
| 30–49 | 42.3 ± 5.1 | 102 | 40.2 | 2.9 | (0.8–10.9) | 0.1 |
| ≧50 | 63.0 ± 8.7 | 195 | 46.2 | 3.7 | (1.0–13.4) | 0.04 |
| GC | 61.6 ± 14.4 | 95 | 64.2 | | | |
| 16–29 | 28 | 1 | 100 | — | — | — |
| 30–49 | 42.4 ± 5.1 | 22 | 63.6 | 1.0^{b} | | |
| ≧50 | 67.9 ± 9.8 | 72 | 63.9 | 1.0 | (0.4–2.7) | 0.9 |
| Gastritis | 53.3 ± 10.1 | 94 | 30.9 | | | |
| 16–29 | — | 0 | — | — | — | — |
| 30–49 | 42.5 ± 4.8 | 35 | 28.6 | 1.0^{b} | | |
| ≧50 | 59.7 ± 6.3 | 59 | 32.2 | 1.2 | (0.5–3.0) | 0.7 |
| DU | 49.4 ± 14.2 | 124 | 35.5 | | | |
| 16–29 | 23.3 ± 3.4 | 15 | 13.3 | 1.0^{b} | | |
| 30–49 | 42.1 ± 5.3 | 45 | 37.8 | 3.9 | (0.8–19.6) | 0.09 |
| ≧50 | 60.6 ± 6.6 | 64 | 39.1 | 4.2 | (0.9–20.0) | 0.07 |

^{a}ORs, 95% CI and P value were performed by logistic regression.
^{b}As the reference to calculate the OR

TABLE III
Characteristics of gastric cancer^{a} analyzed by anti-GroES antibody status

| | Anti-GroES antibody | | |
|---|---|---|---|
| | Positive | Negative | Adjusted OR (95% CI) |
| Variable | no. (%) | no. (%) | P value |
| GENDER | | | |
| Male | 37 (64.9) | 20 (35.1) | 1.0^{d} (0.4–2.5) |
| Female | 24 (63.2) | 14 (36.8) | 0.9 |
| STAGE | | | |
| ^{b}EGC | 12 (70.6) | 5 (29.4) | 1.4^{d} (0.4–4.3) |
| ^{c}AGC | 49 (62.8) | 29 (37.2) | 0.5 |
| Histological type (1) | | | |
| Diffuse | 22 (53.7) | 19 (46.3) | 0.1^{e} |
| Intestinal | 26 (68.4) | 12 (31.6) | |
| Mixed | 4 (100) | 0 (0) | |
| Unclassified | 9 (75) | 3 (25) | |
| Histological type (2) | | | |
| Diffuse | 22 (53.7) | 19 (46.3) | 0.4^{d} (0.2–1.1) |
| Non-diffuse | 39 (72.2) | 15 (27.8) | 0.07 |
| Tumor location (1) | | | |
| Antrum | 46 (71.9) | 18 (28.1) | 0.01^{e} |
| Body | 7 (38.9) | 11 (61.1) | |
| Cardia | 4 (44.4) | 5 (55.6) | |
| Diffuse | 4 (100) | 0 (0) | |
| Tumor location (2) | | | |
| Antrum | 46 (71.9) | 18 (28.1) | 2.7^{d} (1.1–6.7) |
| Non-antrum | 15 (48.4) | 16 (51.6) | 0.03 |

^{a}Gastric cancer serum samples: n = 95
^{b}EGC: early gastric cancer with cancer cell invasion confined to the mucosa or submucosa
^{c}AGC: advanced gastric cancer with cancer cell invasion beyond the muscularis propria
^{d}ORs, 95% CI and P value were performed by logistic regression after controlling for age.
^{e}The P value was obtained using the chi-squared test.

### Example 3

### Induction of Pro-Inflammatory Cytokine Production and COX-2 Expression in PBMC Stimulated with GroES

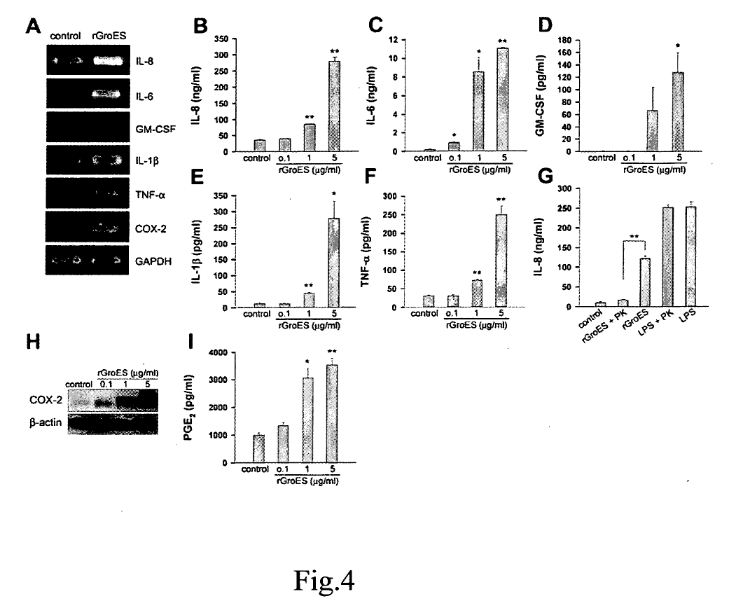

Example 2 demonstrated close association of GroES with GC, a cancer known to result from chronic inflammation caused by H. pylori infection. Moreover, GroES is a secreted protein and in direct contact with host, may mediate important interaction between H. pylori and host. We therefore investigated the effect of GroES on the inflammatory responses of mononuclear cells. PBMC were incubated with rGroES, then mRNA levels for 7 cytokines were determined by RT-PCR. As shown in FIG. 4A, rGroES stimulation caused a marked increase in IL-8, IL-6, IL-1β, and TNF-α, cytokines commonly found in H. pylori-infected patients. In addition, GM-CSF was slightly increased by rGroES (FIG. 4A), while IFN-γ and IL-12 were not changed (data not shown). Furthermore, mRNA levels of COX-2, an enzyme crucial for inflammatory responses, were also greatly enhanced after rGroES stimulation (FIG. 4A). These data showed that H. pylori GroES causes upregulation of the expression of pro-inflammatory cytokines and COX-2 at the transcriptional level.

To correlate the aforementioned increase in mRNA levels with induction of cytokine secretion, we analyzed cytokine protein levels in culture supernatants of PBMC stimulated with rGroES. As shown in FIG. 4B to 4F, rGroES induced a dose-dependent increase in the levels of secreted IL-8, IL-6, GM-CSF, IL-1β, and TNF-α. Induction of cytokine release was seen at concentrations of rGroES as low as 0.1 μg/ml. Stimulation of IL-6 production was almost maximal at 5 μg/ml of rGroES, while secretion of the other cytokines were greatly increasing at this concentration.

To exclude the possibility that the increase in cytokine release induced by rGroES was caused by contaminating LPS, rGroES was digested with proteinase K (PK) before treatment of PBMC and complete digestion was confirmed by the absence of rGroES on silver-stained SDS/PAGE (data not shown). As shown in FIG. 4G, digested materials only caused basal levels of IL-8 production, whereas LPS-induced IL-8 production by PBMC was not affected by PK digestion. These data confirmed that the cytokine production was indeed resulted from stimulation by rGroES instead of LPS.

We also examined the ability of rGroES to induce COX-2 expression at the protein level. As with cytokine production, rGroES induced a dose-dependent increase in COX-2 protein levels in PBMC (FIG. 4H). To confirm this, we examined rGroES-treated PBMC for secretion of PGE₂, whose production depends on COX-2 and is crucial for inflammatory processes. We found that rGroES greatly stimulated PGE₂ release in a dose-dependent manner (FIG. 4I). The level of PGE₂ production was almost saturated at 5 μg/ml of rGroES.

Overall, these results showed that rGroES increases the expression of pro-inflammatory cytokines, COX-2, and PGE₂ at both the transcriptional and translational levels, suggesting that it plays a promoting role in the inflammation triggered by H. pylori infection.

### Example 4

### GroES Induces Production of IL-8, Cell Proliferation, Upregulation of Proto-Oncogenes and Cyclin D1, but Downregulation of p27^{Kip1} in Gastric Epithelial Cells

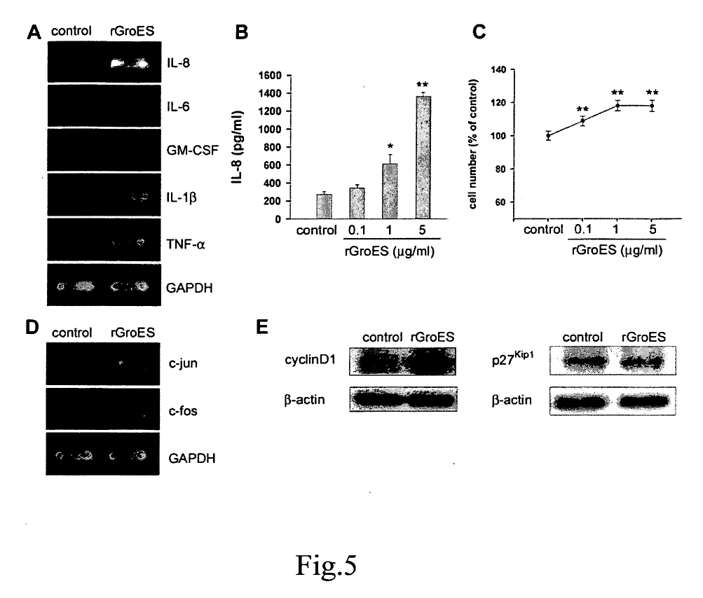

In order to test whether GroES exerted a direct effect on gastric epithelial cells, KATO-III cells, a gastric carcinoma cell line, were treated with rGroES, followed by RT-PCR to determine pro-inflammatory cytokine production. As shown in FIG. 5A, IL-8, GM-CSF, IL-1β, and TNF-α mRNA levels were all increased in rGroES-treated KATO-III cells, while IL-6 mRNA levels were unchanged. Of the 4 cytokines showing increased expression at the transcriptional level, only IL-8 showed a dose-dependent increase in protein secretion (FIG. 5B).

In addition to its promoting role in inflammation, GroES might contribute to GC development by supporting cell proliferation. To test this hypothesis, KATO-III cell proliferation was determined by MTS assay after rGroES stimulation. When treated with 5 μg/ml of rGroES, KATO-III cells significantly increased the number of viable cell up to about 1.2 fold compared with untreated control (FIG. 5C).

Next we used RT-PCR to evaluate the expression of c-jun or c-fos in gastric epithelial cells after rGroES treatment. As shown in FIG. 5D, despite c-jun mRNA was absent and c-fos mRNA were very low in untreated KATO-III cells, the expression of both proto-oncogenes was dramatically increased after rGroES stimulation.

We further examined the supporting role of GroES in GC development by analyzing the protein levels of marker molecules associated with cell cycle regulation. Protein expression of cyclin D1 was upregulated by rGroES (FIG. 5E). Notably, aberrant expression of cyclin D1 has been reported in GC (28). Moreover, we found that p27^{Kip1} protein expression was downregulated by rGroES (FIG. 5E); importantly, reduced expression of p27^{Kip1} is seen in H. pylori-associated intestinal metaplasia (29). Overall, the effect of H. pylori GroES on these cell cycle-related molecules closely matched to those documented in clinical investigations of precancerous gastric lesions and GC.

### Example 5

### H. pylori GroES and FlaG Exhibit Different Effects on Inflammatory Responses and Cell Proliferation

To elucidate the significance of H. pylori GroES in inflammation and cell proliferation, we compared the effects of GroES with the additional H. pylori protein, FlaG (HP0751). FlaG, a polar flagellin, had similar molecular weight to GroES and reacted with low frequency with sera from GC and DU groups (3.1%, n=95 and 12.5%, n=124, respectively). Recombinant FlaG (rFlaG) were also purified and endotoxin-depleted for the treatment of PBMC and KATO-III cells.

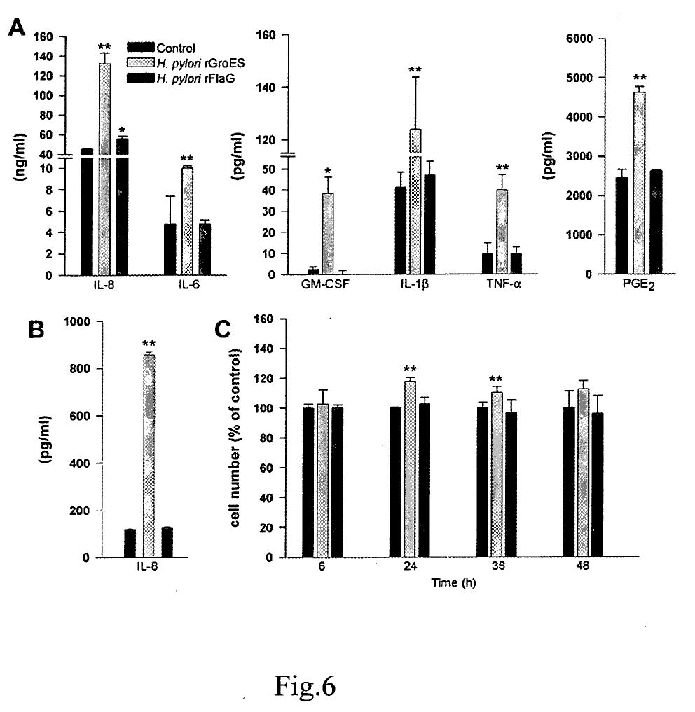

As shown in FIG. 6A, protein levels of IL-8, IL-6, GM-CSF, IL-1β, TNF-α and PGE₂ were highly enhanced by the treatment of rGroES in PBMC. In contrast, rFlaG slightly induced the production of IL-8 in PBMC, but not the other cytokines and PGE₂. In KATO-III cells, IL-8 production was induced much more by rGroES, while rFlaG had no effect on IL-8 production at all (FIG. 6B). We next evaluated the effects of these recombinant proteins on the cell proliferation in KATO-III cells. As shown in FIG. 6C, cell number was significantly increased when incubation with rGroES for 24-36 h. In contrast, rFlaG had no effect on cell proliferation.

According to aforesaid examples, GroES protein of H. pylori is highly related with GC patients in clinical serology. A serological study in example 2 showed that 64.2% of GC sera reacted with H. pylori GroES compared to 30.9% of gastritis samples and 35.5% of DU samples, and that there was no significant difference in GroES seropositivity between the early and advanced stages of GC. Notably, our results of prevalence survey were different from those reported by three other groups, who found that GroES seropositivity among H. pylori-infected adults increased gradually with age in developed countries and in a developing country, Mexico (30-32). In addition, Pérez-Pérez et al. reported that the incidence of GroES seropositivity is high in adenocarcinoma of the cardia, a lesion not associated with H. pylori infection (30), while Ng et al. showed that GroES antigenicity is not related to the clinical outcome of H. pylori infection (31). In contrast to their findings, the present invention demonstrated that GroES seropositivity was closely associated with antral GC, a non-cardia cancer associated with H. pylori infection (3). Furthermore, according to example 3, rGroES can induce the production of pro-inflammatory cytokines, including IL-8, IL-6, GM-CSF, IL-1β, and TNF-α with does-dependent increase. Even though the concentration of rGroES is as low as 0.1 μg/ml, cytokines are still induced to release. Specifically, IL-6 is a multifunctional cytokine that functions as growth and differentiation factor for tumor cells (33). IL-8 has been proposed to act as a promoter of tumor growth through its angiogenic properties (34). GM-CSF and IL-1β are also potent growth factors for gastric epithelial cells (35). IL-11 and TNF-α are powerful inhibitors of gastric acid secretion (36) It is known that reduced acid secretion leads to increased levels of gastrin and thus provides continuous proliferating stimuli to gastric epithelial cells (37), and the subsequent atrophic changes may lead to an increased risk of non-cardia carcinogenesis (8). Therefore, applicants have proved GroES in the present invention is a virulent factor related to induce the production of proinflammatory cytokines. Moreover, it indicated that GroES might promote inflammation by enhancing. COX-2 expression in PBMC, leading to the production of PGE₂, which is known to participate in the inflammatory process, inhibition of apoptosis, angiogenesis, and tumorigenesis (38-41).

In the example 4, it's also suggested a positive effect of H. pylori GroES on the growth of gastric epithelial cells by enhancing the expression of proteins associated with cell proliferation. We found that GroES induced expression of c-jun, c-fos, and cyclin D1, while p27^{Kip1} protein was down-regulated.

In conclusion, the present invention utilizes a comparison of responses of serum antibodies from GC and DU patients to the H. pylori proteome leads to the identification of GroES as a dominant GC-associated antigen of H. pylori. We further demonstrate that GroES seropositivity is highly associated with antral GC, suggesting its value as a prediction marker for GC. Moreover, a novel role for H. pylori GroES in the development of GC is established, which appears to involve the inflammation induced by H. pylori infection and the promotion of molecular changes favoring cell proliferation. Furthermore, taking the nucleic acid or amino acid of GroES as biomarkers to detect H. pylori-related gastric disease or GC will be helpful to clinical diagnosis and treatment.

### Other Embodiments

All features disclosed herein may be combined in any form with other methods and replaced by other features with identical, equivalent or similar purpose. Thus except for the part that is specifically emphasized, all features disclosed herein constitute only one embodiment among the numerous equivalent or similar features.

All modifications and alterations to the descriptions disclosed herein made by those skilled in the art without departing from the spirits of the invention and appended claims shall remain within the protected scope and claims of the invention.

### REFERENCES

1. Marshall, B. and Warren, J. R. (1983) Unidentified curved bacilli on gastric epithelium in active chronic gastritis. Lancet 1, 1273-1275.
2. Peterson, W. L. (1991) Helicobacter pylori and peptic ulcer disease. N. Engl. J. Med. 324, 1043-1048.
3. Parsonnet, J., Friedman, G. D., Vandersteen, D. P., Chang, Y., Vogelman, J. H., Orentreich, N., and Sibley, R. K. (1991) Helicobacter pylori infection and the risk of gastric carcinoma. N. Engl. J. Med. 325, 1127-1131.
4. Nomura, A., Stemmermann, G. N., Chyou, P. H., Kato, I., Perez-Perez, G. I., and Blaser, M. J. (1991) Helicobacter pylori infection and gastric carcinoma among Japanese Americans in Hawaii. N. Engl. J. Med. 325, 1132-1136.
5. Murray, C. J. and Lopez, A. D. (1997) Alternative projections of mortality and disability by cause 1990-2020: Global Burden of Disease Study. Lancet 349, 1498-1504.
6. Uemura, N., Okamoto, S., Yamamoto, S., Matsumura, N., Yamaguchi, S., Yamakido, M., Taniyama, K., Sasaki, N., and Schlemper, R. J. (2001) Helicobacter pylori infection and the development of gastric cancer. N. Engl. J. Med. 345, 784-789.
7. Ekstrom, A. M., Held, M., Hansson, L. E., Engstrand, L., and Nyren, O. (2001) Helicobacter pylori in gastric cancer established by CagA immunoblot as a marker of past infection. Gastroenterology 121, 784-791.
8. Blaser, M. J. and Atherton, J. C. (2004) Helicobacter pyloripersistence: biology and disease. J. Clin. Invest 113, 321-333.
9. Hansson, L. E., Nyren, O., Hsing, A. W., Bergstrom, R., Josefsson, S., Chow, W. H., Fraumeni, J. F., Jr., and Adami, H. O. (1996) The risk of stomach cancer in patients with gastric or duodenal ulcer disease. N. Engl. J. Med. 335, 242-249.
10. Suerbaum, S. and Michetti, P. (2002) Helicobacter pylori infection. N. Engl. J. Med. 347, 1175-1186.
11. Haas, G., Karaali, G., Ebermayer, K., Metzger, W. G., Lamer, S., Zimny-Arndt, U., Diescher, S., Goebel, U. B., Vogt, K., Roznowski, A. B., Wiedenmann, B. J., Meyer, T. F., Aebischer, T., and Jungblut, P. R. (2002) Immunoproteomics of Helicobacter pylori infection and relation to gastric disease. Proteomics. 2, 313-324.
12. Krah, A., Miehlke, S., Pleissner, K. P., Zimny-Arndt, U., Kirsch, C., Lehn, N., Meyer, T. F., Jungblut, P. R., and Aebischer, T. (2004) Identification of candidate antigens for serologic detection of Helicobacter pylori-infected patients with gastric carcinoma. Int. J. Cancer 108, 456-463.
13. Harris, P. R., Smythies, L. E., Smith, P. D., and Dubois, A. (2000) Inflammatory cytokine mRNA expression during early and persistent Helicobacter pylori infection in nonhuman primates. J. Infect. Dis. 181, 783-786.
14. Crabtree, J. E., Shallcross, T. M., Heatley, R. V., and Wyatt, J. I. (1991) Mucosal tumour necrosis factor alpha and interleukin-6 in patients with Helicobacter pylori associated gastritis. Gut 32, 1473-1477.
15. Noach, L. A., Bosma, N. B., Jansen, J., Hoek, F. J., van Deventer, S. J., and Tytgat, G. N. (1994) Mucosal tumor necrosis factor-alpha, interleukin-1 beta, and interleukin-8 production in patients with Helicobacter pylori infection. Scand. J. Gastroenterol. 29, 425-429.
16. Crabtree, J. E., Covacci, A., Farmery, S. M., Xiang, Z., Tompkins, D. S., Perry, S., Lindley, I. J., and Rappuoli, R. (1995) Helicobacter pylori induced interleukin-8 expression in gastric epithelial cells is associated with CagA positive phenotype. J. Clin. Pathol. 48, 41-45.
17. Kabir, S. and Daar, G. A. (1995) Serum levels of interleukin-1, interleukin-6 and tumour necrosis factor-alpha in patients with gastric carcinoma. Cancer Lett. 95, 207-212.
18. Kitadai, Y., Haruma, K., Sumii, K., Yamamoto, S., Ue, T., Yokozaki, H., Yasui, W., Ohmoto, Y., Kajiyama, G., Fidler, I. J., and Tahara, E. (1998) Expression of interleukin-8 correlates with vascularity in human gastric carcinomas. Am. J. Pathol. 152, 93-100.
19. El-Omar, E. M., Rabkin, C. S., Gammon, M. D., Vaughan, T. L., Risch, H. A., Schoenberg, J. B., Stanford, J. L., Mayne, S. T., Goedert, J., Blot, W. J., Fraumeni, J. F., Jr., and Chow, W. H. (2003) Increased risk of noncardia gastric cancer associated with proinflammatory cytokine gene polymorphisms. Gastroenterology 124, 1193-1201.
20. El-Omar, E. M., Carrington, M., Chow, W. H., McColl, K. E., Bream, J. H., Young, H. A., Herrera, J., Lissowska, J., Yuan, C. C., Rothman, N., Lanyon, G., Martin, M., Fraumeni, J. F., Jr., and Rabkin, C. S. (2000) Interleukin-1 polymorphisms associated with increased risk of gastric cancer. Nature 404, 398-402.
21. Kai, H., Kitadai, Y., Kodama, M., Cho, S., Kuroda, T., Ito, M., Tanaka, S., Ohmoto, Y., and Chayama, K. (2005) Involvement of proinflammatory cytokines IL-1beta and IL-6 in progression of human gastric carcinoma. Anticancer Res. 25, 709-713.
22. Eguchi, H., Herschenhous, N., Kuzushita, N., and Moss, S. F. (2003) Helicobacter pylori increases proteasome-mediated degradation of p27 (kip1) in gastric epithelial cells. Cancer Res. 63, 4739-4746.
23. Meyer-ter-Vehn, T., Covacci, A., Kist, M., and Pahl, H. L. (2000) Helicobacter pylori activates mitogen-activated protein kinase cascades and induces expression of the proto-oncogenes c-fos and c-jun. J. Biol. Chem. 275, 16064-16072.
24. Hirata, Y., Maeda, S., Mitsuno, Y., Akanuma, M., Yamaji, Y., Ogura, K., Yoshida, H., Shiratori, Y., and Omata, M. (2001) Helicobacter pylori activates the cyclin D1 gene through mitogen-activated protein kinase pathway in gastric cancer cells. Infect. Immun. 69, 3965-3971.
25. Lauren, P. (1965) The two histological main types of gastric carcinoma: diffuse and so-called intestinal-type carcinoma. An attempt at a histo-clinical classification. Acta Pathol. Microbiol. Scand. 64, 31-49.
26. Utt, M., Nilsson, I., Ljungh, A., and Wadstrom, T. (2002) Identification of novel immunogenic proteins of Helicobacter pylori by proteome technology. J. Immunol. Methods 259, 1-10.
27. Kao, S. H., Su, S. N., Huang, S. W., Tsai, J. J., and Chow, L. P. (2005) Sub-proteome analysis of novel IgE-binding proteins from Bermuda grass pollen. Proteomics. 5, 3805-3813.
28. Gao, P., Zhou, G. Y., Liu, Y., Li, J. S., Zhen, J. H., and Yuan, Y. P. (2004) Alteration of cyclin D1 in gastric carcinoma and its clinicopathologic significance. World J. Gastroenterol. 10, 2936-2939.
29. Yu, J., Leung, W. K., Ng, E. K., To, K. F., Ebert, M. P., Go, M. Y., Chan, W. Y., Chan, F. K., Chung, S. C., Malfertheiner, P., and Sung, J. J. (2001) Effect of Helicobacter pylori eradication on expression of cyclin D2 and p27 in gastric intestinal metaplasia. Aliment. Pharmacol. Ther. 15, 1505-1511.
30. Perez-Perez, G. I., Thiberge, J. M., Labigne, A., and Blaser, M. J. (1996) Relationship of immune response to heat-shock protein A and characteristics of Helicobacter pylori-infected patients. J. Infect. Dis. 174, 1046-1050.
31. Ng, E. K., Thompson, S. A., Perez-Perez, G. I., Kansau, I., van der, E. A., Labigne, A., Sung, J. J., Chung, S. C., and Blaser, M. J. (1999) Helicobacter pylori heat shock protein A: serologic responses and genetic diversity. Clin. Diagn. Lab Immunol. 6, 377-382.
32. Eamranond, P. P., Torres, J., Munoz, O., and Perez-Perez, G. I. (2004) Age-specific immune response to HspA in Helicobacter pylori-positive persons in Mexico. Clin. Diagn. Lab Immunol. 11, 983-985.
33. Onozaki, K., Akiyama, Y., Okano, A., Hirano, T., Kishimoto, T., Hashimoto, T., Yoshizawa, K., and Taniyama, T. (1989) Synergistic regulatory effects of interleukin 6 and interleukin 1 on the growth and differentiation of human and mouse myeloid leukemic cell lines. Cancer Res. 49, 3602-3607.
34. Kitadai, Y., Takahashi, Y., Haruma, K., Naka, K., Sumii, K., Yokozaki, H., Yasui, W., Mukaida, N., Ohmoto, Y., Kajiyama, G., Fidler, I. J., and Tahara, E. (1999) Transfection of interleukin-8 increases angiogenesis and tumorigenesis of human gastric carcinoma cells in nude mice. Br. J. Cancer 81, 647-653.
35. Beales, I. L. (2002) Effect of interleukin-1beta on proliferation of gastric epithelial cells in culture. BMC. Gastroenterol. 2, 7.
36. Beales, I. L. and Calam, J. (1998) Interleukin 1 beta and tumour necrosis factor alpha inhibit acid secretion in cultured rabbit parietal cells by multiple pathways. Gut 42, 227-234.
37. Peek, R. M., Jr., Wirth, H. P., Moss, S. F., Yang, M., Abdalla, A. M., Tham, K. T., Zhang, T., Tang, L. H., Modlin, I. M., and Blaser, M. J. (2000) Helicobacter pylori alters gastric epithelial cell cycle events and gastrin secretion in Mongolian gerbils. Gastroenterology 118, 48-59.
38. Seibert, K., Zhang, Y., Leahy, K., Hauser, S., Masferrer, J., Perkins, W., Lee, L., and Isakson, P. (1994) Pharmacological and biochemical demonstration of the role of cyclooxygenase 2 in inflammation and pain. Proc. Natl. Acad. Sci. U.S. A 91, 12013-12017.
39. Boolbol, S. K., Dannenberg, A. J., Chadburn, A., Martucci, C., Guo, X. J., Ramonetti, J. T., breu-Goris, M., Newmark, H. L., Lipkin, M. L., DeCosse, J. J., and Bertagnolli, M. M. (1996) Cyclooxygenase-2 overexpression and tumor formation are blocked by sulindac in a murine model of familial adenomatous polyposis. Cancer Res. 56, 2556-2560.
40. Kim, J. M., Kim, J. S., Jung, H. C., Song, I. S., and Kim, C. Y. (2000) Upregulated cyclooxygenase-2 inhibits apoptosis of human gastric epithelial cells infected with Helicobacter pylori. Dig. Dis. Sci. 45, 2436-2443.
41. Leung, W. K., To, K. F., Go, M. Y., Chan, K. K., Chan, F. K., Ng, E. K., Chung, S.C., and Sung, J. J. (2003) Cyclooxygenase-2 upregulates vascular endothelial growth factor expression and angiogenesis in human gastric carcinoma. Int. J. Oncol. 23, 1317-1322.

What is claimed is:

1. A biomarker for detecting gastric diseases selected from:
a nucleic acid sequence of GroES, complementary strand, or derivatives thereof or an amino acid sequence of GroES, derivatives, fragments or variants thereof or antibodies against said amino acid sequences or combinations thereof.

2. The biomarker as claimed in claim 1, wherein said GroES is a specific protein of H. pylori.

3. The biomarker as claimed in claim 1, wherein said nucleic acid sequence of GroES is SEQ ID NO:1.

4. The biomarker as claimed in claim 1, wherein said amino acid sequence of GroES is SEQ ID NO:2.

5. The biomarker as claimed in claim 1, wherein said variants have more than 80% similarity with the amino acid sequence of SEQ ID NO:2.

6. The biomarker as claimed in claim 1, wherein said derivatives means the nucleic acid or the complementary strand which 3′ or 5′ terminal was modified with other nucleic acid to show sequence homology with SEQ ID NO:1 greater than 90%.

7. The biomarker as claimed in claim 1, wherein said gastric diseases is gastric cancer.

8. A biomarker for detecting gastric diseases selected from: a nucleic acid sequence of SEQ ID NO:1, complementary strand, derivatives thereof or an amino acid sequence of SEQ ID NO:2, derivatives, fragments, variants thereof or antibodies against said amino acid sequences or combinations thereof.

9. The biomarker as claimed in claim 8, wherein said variants have more than 80% similarity with the amino acid sequence of SEQ ID NO:2.

10. The biomarker as claimed in claim 8, wherein said derivatives means the nucleic acid or the complementary strand which 3′ or 5′ terminal was modified with other nucleic acid to show sequence homology greater than 90%.

11. A kit for detecting gastric disease, comprising a biomarker selected from: a nucleic acid sequence of GroES, complementary strand or derivatives thereof or an amino acid sequence of GroES, derivatives, fragments or variants thereof or antibodies against said amino acid sequences or combinations thereof.

12. The kit as claimed in claim 11, wherein said GroES is a specific protein of H. pylori.

13. The kit as claimed in claim 11, wherein sequence in said nucleic acid of GroES is SEQ ID NO:1.

14. The kit as claimed in claim 11, wherein said amino acid sequence of GroES is SEQ ID NO:2.

15. The kit as claimed in claim 11, further comprising a second antibody which can recognize any amino acid sequences of SEQ ID NO:2, derivatives, fragments, variants thereof or secondary antibodies against said amino acid sequences or combinations thereof.

16. A method for detecting gastric cancer, comprising
(a) providing samples;
(b) providing biomarkers, selected from: a nucleic acid showing SEQ ID NO:1, complementary strand, or derivatives thereof or an amino acid sequence showing SEQ ID NO:2, derivatives, fragments or variants thereof or antibodies against said amino acid sequences or combinations thereof;
(c) contacting said biomarkers with said samples, wherein said analytes selected from: a nucleic acid sequence of GroES, complementary strand or derivatives thereof or an amino acid sequence of GroES, derivatives, fragments or variants thereof or antibodies against said amino acid sequences or combinations thereof;
(d) detecting products which result from said biomarkers contacting with said analytes in said step (c).

17. The method as claimed in claim 16, wherein said samples are serum, saliva or stomach tissue.

18. The method as claimed in claim 16, wherein said biomarkers are further immobilized on substrate.

19. The method as claimed in claim 18, wherein said substance is membrane, microplates or biochips.

20. The method as claimed in claim 16, wherein said analytes in said samples are further labelled with fluorescence markers.

21. The method as claimed in claim 16, further comprising a step for utilizing secondary antibody to recognize corresponding antibody before step (d).

22. The method as claimed in claim 16, wherein said detecting product in step (d) by means of ELISA (enzyme-linked immunosorbent assay), RIA (radioimmunoassay), western blot or immunofluorescence assay.

23. The method as claimed in claim 16, wherein said detecting product in step (d) by means of RT-PCR (reverse transcriptase-polymerase chain reaction) or in situ hybridization.

24. A biomarker for detecting gastric cancer, wherein said biomarker is selected from: an amino acid sequence of GroES, derivatives, fragments, variants thereof or the antibodies against said amino acid sequences or combinations thereof.

25. The biomarker as claimed in claim 24, wherein said GroES is a specific protein of H. pylori.

26. The biomarker as claimed in claim 24, wherein said amino acid sequence of GroES is SEQ ID NO:2.

27. The biomarker as claimed in claim 24, wherein said variants have more than 80% similarity with the amino acid sequence of SEQ ID NO:2.

28. A kit for detecting gastric cancer, comprising a biomarker selected from: an amino acid sequence of GroES, derivatives, fragments, variants thereof or the antibodies against said amino acid sequences or combinations thereof, wherein said GroES is a specific protein of H. pylori.

29. The kit as claimed in claim 28, wherein said amino acid sequence of GroES is SEQ ID NO:2.

30. The kit as claimed in claim 28, further comprising a second antibody which can recognize any amino acid sequences of SEQ ID NO:2, derivatives, fragments, variants thereof or antibodies against said amino acid sequences or combinations thereof.


SEQUENCE LISTING (2 sequences)

SEQ ID NO: 1 - DNA, 357 bp, Helicobacter pylori
atgaagtttcagccattaggagaaagggtcttagtagaaagacttgaagaagagaacaaaaccagttcaggcatcatcatccctgataacgctaaggaaaagcctttaatgggcgtagtcaaagcggttagccataaaatcagtgagggttgcaaatgcgttaaagaaggcgatgtgatcgcttttggcaaatacaaaggcgcagaaatcgttttagacggcactgaatacatggtgctagaactagaagacattctaggcattgtgggctcaggctcttgttgtcatacaggtaatcatgaccataaacatgctaaagagcatgaagcttgctgtcatgatcacaaaaaacactaa

SEQ ID NO: 2 - protein, 118 aa, Helicobacter pylori
MKFQPLGERVLVERLEEENKTSSGIIIPDNAKEKPLMGVVKAVSHKISEGCKCVKEGDVIAFGKYKGAEIVLDGTEYMVLELEDILGIVGSGSCCHTGNHDHKHAKEHEACCHDHKKH